(19) United States
(12) Patent Application Publication
(10) Pub. No.: US 2017/0312343 A1
(43) Pub. Date: Nov. 2, 2017

(54) Uses of FAHD1

(71) Applicants: UNIVERSITÄT INNSBRUCK, Innsbruck (AT); ÖSTERREICHISCHE AKADEMIE DER WISSENSCHAFTEN, Wien (AT)

(72) Inventors: Eva Albertini, Innsbruck (AT); Elisabeth Mayr, Hall (AT); Andrea Taferner, Innsbruck (AT); Haymo Pircher, Zirl (AT); Pidder Jansen-Duerr, Innsbruck (AT); Susanne von Grafenstein, Innsbruck (AT); Christian Kramer, Lörrach (DE); Klaus R. Liedl, Mils (AT); Thomas Diener, Kundl (AT); Christina Metzger, Innsbruck (AT)

(21) Appl. No.: 15/523,507

(22) PCT Filed: Oct. 30, 2015
(86) PCT No.: PCT/EP2015/075362

§ 371 Date: May 1, 2017

(30) Foreign Application Priority Data
Oct. 31, 2014 (EP) 14191224.6

(51) Int. Cl. A61K 38/46; C12P 7/40; C12Q 1/34

(52) U.S. Cl. CPC A61K 38/46; C12Y 307/01005; C12Q 1/34; C12P 7/40; G01N 2500/02; G01N 2500/20

(57) ABSTRACT

The present invention provides FAHD1 for use in a method for the treatment or prevention of aberrations of the energy metabolism of the nervous system, pancreas, kidney, liver, muscles or adipose tissue. Further, a method of decarboxylating an organic compound is provided, which uses FAHD1 to decarboxylate the organic compound. Additionally, a method and a kit for identifying inhibitors of FAHD1 are provided.

The present invention provides FAHD1 for use in a method for the treatment or prevention of aberrations of the energy metabolism of the nervous system, pancreas, kidney, liver, muscles or adipose tissue.

Further, a method of decarboxylating an organic compound is provided, which uses FAHD1 to decarboxylate the organic compound. Moreover, a method and a kit for identifying inhibitors of FAHD1 are provided.

In eukaryotes, carbohydrates and several amino acids are degraded in parallel pathways converging at the generation of pyruvate, which is imported into the mitochondria for subsequent oxidation. The pyruvate dehydrogenase complex converts pyruvate to acetyl-CoA, which is condensed with oxaloacetate to produce citrate, comprising the first step of the tricarboxylic acid (TCA) cycle, also called citric acid cycle (CAC) or Krebs cycle. The TCA cycle, which takes place inside mitochondria in eukaryotic cells, accounts for about two-thirds of the total oxidation of carbon compounds in most cells and is thus at the center of the energy metabolism of the cells. One major intermediate of the TCA cycle is oxaloacetate onto which acetyl groups from acetyl-CoA are transferred to form citric acid. Additionally, the TCA cycle also functions as a starting point for important biosynthetic reactions by providing vital carbon-containing intermediates like oxaloacetate and α-ketoglutarat. Sustained metabolic flux through the TCA cycle thus requires sufficient supply of oxaloacetate, the concentration of which decreases in response to distinct metabolic changes, e.g. the withdrawal of intermediate TCA cycle metabolites for biosynthetic processes. Under these conditions, an anaplerotic pathway is activated where pyruvate is directly converted to oxaloacetate (Jitrapakdee et al., Cell Mol Life Sci, 2006; 63(7-8)). The enzyme responsible for this reaction and thus for replenishing oxaloacetate in the TCA cycle is pyruvate carboxylase (PC), a regulated mitochondrial enzyme that catalyzes the conversion of pyruvate to oxaloacetate. The enzymatic activity of PC thus facilitates flux through a key intermediary metabolism reaction, i.e. the ATP-dependent carboxylation of pyruvate to oxaloacetate. Accordingly, the molar ratio between pyruvate and oxaloacetate regulates cellular energy metabolism, with PC maintaining the equilibrium between both metabolites. Enzymes carrying out the inverse reaction, i.e. decarboxylation of oxaloacetate, may furthermore modulate mitochondrial metabolism of pyruvate. While oxaloacetate decarboxylases (ODx) exist in several bacteria where they fulfill specific metabolic tasks related to survival in particular ecological niches, dedicated ODx enzymes were not identified in eukaryotes up to now. However, elevated levels of oxaloacetate inhibit essential mitochondrial enzymes, suggesting that the concentration of oxaloacetate in mitochondria must be tightly regulated. Still, mechanisms to specifically reduce the concentration of oxaloacetate within mitochondria via decarboxylation remain elusive.

The reaction catalyzed by PC constitutes the best recognized interconversion required for the replenishment of pools of intermediates of the TCA cycle, a process named anaplerosis that restores losses of TCA cycle derivative products which occur during normal metabolism. As an anaplerotic enzyme, PC participates in metabolic pathways depending on the availability of oxaloacetate such as gluconeogenesis, glycogensynthesis, lipogenesis, glyceroneogenesis, the synthesis of non-essential amino acids and neurotransmitters as well as glucose-dependent insulin secretion. Because PC is essential for these interrelated aspects of anabolism, deficiency of this enzyme can cause metabolic disturbances in numerous tissues whose metabolism depends upon high TCA cycle flux, like liver, kidney, brain, pancreas, muscles and adipose tissue.

Given the diverse roles of PC in energy metabolism and the importance of oxaloacetate as an intermediate of the TCA cycle as well as different biosynthetic pathways, therapeutic agents are needed which are able to affect oxaloacetate levels and thus treat aberrations of the energy metabolism, in particular the lipid, glucose and amino acid metabolism. The technical problem underlying the present invention is thus to provide agents and means to prevent and treat aberrations of the energy metabolism.

This problem is solved by the subject-matter of the independent claims, in particular by the provision of FAHD1 (fumarylacetoacetate hydrolase domain containing protein 1) for use in a method for the prevention or treatment of aberrations of the energy metabolism of the nervous system, pancreas, kidney, liver, muscles or adipose tissue.

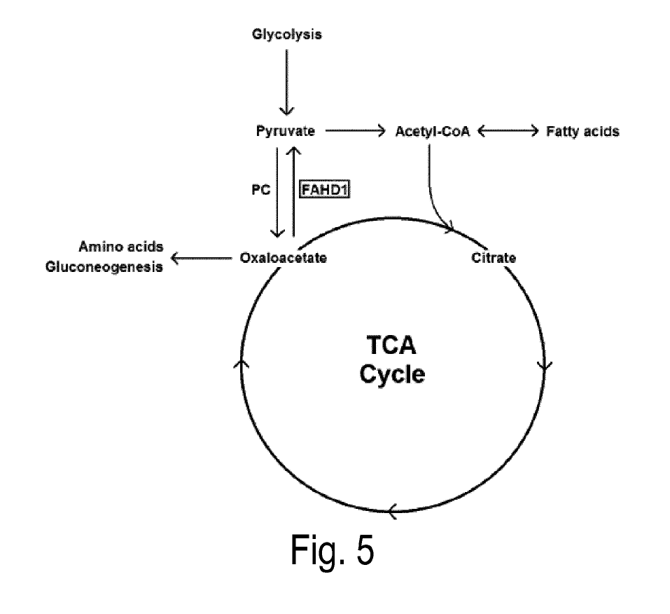

The present inventors surprisingly found that mammalian, in particular human or murine, fumarylacetoacetate hydrolase domain containing protein 1 (FAHD1), belonging to the FAH (fumarylacetoacetate hydrolase) domain containing protein family, possesses oxaloacetate decarboxylase activity and thus is a PC antagonist catalyzing the decarboxylation of oxaloacetate to pyruvate. The FAH domain containing protein family is a superfamily of metabolic enzymes with a very diverse set of catalytic activities. In the prior art, FAHD1 was characterized as an enzyme having an acylpyruvase activity based on the ability of the recombinant protein to catalyze the hydrolysis of acylpyruvates such as fumarylpyruvate (Pircher et al., J Biol Chem, 2011, 286(42)). However, based on the present results, FAHD1 functions as an ODx enzyme in vivo. FAHD1 and PC thus form a metabolic module, in particular an equilibrium metabolic module, balancing the concentrations of oxaloacetate and pyruvate. The respective reactions are depicted in FIG. 5.

Thus, by employing the present teaching characterizing the decarboxylating enzymatic activity of FAHD1, the present invention provides means and methods to reduce the concentration of oxaloacetate in mammals, in particular within mitochondria of mammals. The present invention also provides means and methods to counteract the activity of PC in mammals, in particular in mitochondria of mammals. Further, the present invention provides means and methods to increase the level of pyruvate in mammals, in particular in mitochondria of mammals.

In the context of the present invention, energy metabolism is understood to mean the metabolism of lipids, carbohydrates, especially glucose, and amino acids. Preferably, in the present context the term energy metabolism means oxaloacetate metabolism, and in a furthermore preferred embodiment, relates to a reaction wherein oxaloacetate is a precursor or starting material and/or an intermediate or final product of the reaction. Thus, in a particularly preferred embodiment, the energy metabolism involves the provision or reduction of oxaloacetate, in particular its concentration.

The present invention provides the teaching that FAHD1 is able to decarboxylate oxaloacetate in vitro and in vivo. In particular, it was found that oxaloacetate levels are increased in liver and kidney derived from FAHD1 knockout mice, which evidences the role of oxaloacetate as in vivo substrate of FAHD1. The present invention further provides the teaching that FAHD1 antagonizes the activity of pyruvate carboxylase (PC) which is known to catalyze the synthesis of oxaloacetate from pyruvate and CO₂. Thus, the present invention teaches the existence of a regulatory network or module linking PC and FAHD1. Since in healthy subjects this module functions to keep the intercellular concentration of oxaloacetate in the physiological range and to adapt the oxaloacetate supply to the changing metabolic needs of said organism, the present invention provides for means and methods to treat imbalances in the energy metabolism, in particular metabolic pathways relying on PC and FAHD1 activity, aberrations of which are associated with several severe diseases.

The provision of oxaloacetate is also called anaplerosis. In an anaplerotic reaction intermediates, presently oxaloacetate, of a metabolic pathway, in particular the TCA cycle, are formed to keep the concentration of the TCA cycle intermediates constant. The reduction of the oxaloacetate concentration is also called anti-anaplerosis or cataplerosis. An anti-anaplerotic or cataplerotic reaction relates to a reaction extracting a TCA cycle intermediate from said cycle for biosynthesis. Thus, energy metabolism in the present context relates to the provision and regulation of oxaloacetate levels for the TCA cycle and biosynthetic pathways depending on oxaloacetate, such as gluconeogenesis, fatty acid synthesis, glyceroneogenesis, insulin secretion and synthesis of neurotransmitters, e.g. glutamate.

In the context of the present invention, the term “aberrations” of the energy metabolism means disruptions, deficiencies or diseases concerning the energy metabolism. Thus, the energy metabolism according to the present invention is in a preferred embodiment the provision of oxaloacetate for gluconeogenesis, de novo fatty acid synthesis, glyceroneogenesis, insulin secretion and de novo synthesis of neurotransmitters, e.g. glutamate.

In a preferred embodiment of the present invention, the aberration of the energy metabolism is type 2 diabetes mellitus, obesity, hypercholesterolemia, metabolic disease, epilepsy, attention deficit hyperactivity disorder (ADHD), Parkinson's disease, Alzheimer's disease, focal cerebral ischemia (stroke), lactic acidosis, psychomotor deficiencies, mental disorder or death in infancy.

PC is known to be involved in carbohydrate and lipid metabolism, e.g. by the highly regulated expression in gluconeogenic tissues, adipose tissues and pancreatic islets depending on the nutrition state. Thus, the counteracting FAHD1 enzyme represents a potential effector for a method for the prevention or treatment of aberrations of the energy metabolism of the nervous system, pancreas, kidney, liver, muscles or adipose tissue.

Clinical manifestations of aberration of the energy metabolism are for example type 2 diabetes mellitus, obesity, hypercholesterolemia, metabolic syndrome, epilepsy, attention deficit hyperactivity disorder (ADHD), Parkinson's disease, Alzheimer's disease, focal cerebral ischemia (stroke), lactic acidosis, psychomotor deficiencies, mental disorders or death in infancy.

Experiments with C. elegans mutants with depletion of FAHD-1, the nematode homologue of FAHD1 (fahd-1(tm5005) mutants), showed severe locomotion defects and strongly reduced capacity for physical activity. Thus, there is an established relation between enhanced oxaloacetate levels and locomotion deficiencies. Synthesis pathways of the neurotransmitter glutamate and other neurotransmitters depend on the provision of oxaloacetate as precursor. It is known that different neurological disease or mental disorders are associated with imbalances in neurotransmitter levels. Enhancing ODx activity by provision of FAHD1 is regarded as potential tool in manipulating neurotransmitter ratios, i.e. by decreasing glutamate synthesis via down regulating the concentration of the precursor oxaloacetate. Thus, it is believed that FAHD1 or homologues thereof are suitable for use in treatment or therapy of aberrations of the nervous system, such as epilepsy, attention deficit hyperactivity disorder (ADHD), Parkinson's disease, Alzheimer's disease, focal cerebral ischemia (stroke), psychomotor deficiencies, or mental disorders. In this context mental disorders comprise for example depression or schizophrenia.

The relation between FAHD1 and metabolic regulation mechanisms is further proven by the regulation of FAHD1 gene expression dependent on the nutrition state in mice. In the kidney an increase of expression of mRNA encoding FAHD1 was observed upon fasting. Although the increase is not as prominent as observed for the mRNA encoding enzymes pyruvate carboxylase or phosphoenolpyruvate carboxykinase, it indicates the connection between FAHD1 and energy metabolism. In the liver, the expression profiles of mRNA encoding the enzymes pyruvate carboxylase and phosphoenolpyruvate carboxykinase also showed a significant increase upon fasting and decreased upon resupply of nutrition. In contrast, for FAHD1 in liver, the trend opposed to the other two investigated proteins during feeding/fasting cycle. The level of FAHD1 expression in liver decreased slightly after fasting and a significant increase is observed upon re-feeding of the investigated mice. Thus, FAHD1 is regulated dependent on the nutrition state. The differentiated expression patterns in the investigated organs suggest a complex role of FAHD1 in metabolic pathways.

Wild-type mice were compared with FAHD1 knock out mice and no visible alterations in the phenotypes were observable at young age. However, some of the investigated clinical chemistry parameters related to fat metabolism revealed interesting differences between both groups. Knockout mice showed decreased cholesterol levels after overnight fasting, whereas glycerol levels were slightly increased compared to wild-type mice. Most interestingly, the amount of HDL-cholesterol was significantly decreased in knockout mice (p=0.016). Low HDL-cholesterol is associated with metabolic syndrome. As lack of FAHD1 results in signs of metabolic syndrome, supply of FAHD1 should have beneficial effects on HDL-cholesterol levels and thus is useful in prevention or treatment of metabolic syndrome and also other diseases associated to low HDL-cholesterol levels, e.g. hypercholesterolemia, dyslipoproteinemia, type 2 diabetes mellitus, or arteriosclerotic vascular disease.

Thus, in a preferred embodiment of the present invention, metabolic syndrome, the aberration of the energy metabolism is metabolic syndrome.

The present invention further provides the use of FAHD1 or a homologue thereof, which homologue comprises an amino acid sequence with at least 80% identity to FAHD1, in a method for the prevention or treatment of aberrations of the energy metabolism of the nervous system, pancreas, kidney, liver, muscles or adipose tissue.

Thus, in this case aberration of energy metabolism relates to the reduction of oxaloacetate, in particular its concentration.

In a preferred embodiment of the present invention, the FAHD1 protein is used in a method of the prevention or treatment of aberrations of the energy metabolism of the nervous system, pancreas, kidney, liver, muscles or adipose tissue.

In a further preferred embodiment, the FAHD1 gene is used to increase FAHD1 protein via gene therapy to treat or prevent aberrations of the energy metabolism of the nervous system, pancreas, kidney, liver, muscles or adipose tissue.

In the context of the present invention, FAHD1 is a fumarylacetoacetate hydrolase domain containing protein 1, preferably from human, such as described in PDB database code 1SAW and Manjasetty et al., Biol Chem, 2004, 385(10) which is incorporated herein by reference.

In the context of the present invention, a homologue of FAHD1, which comprises an amino acid sequence with at least 80% identity to FAHD1, in particular to human FAHD1, is a protein with an amino acid sequence identity as determined by the method of Altschul et al. (1990, J Mol Biol, 215(3) and Nucl. Acids Res., 1997, 25 (17)) of at least 80% identity, preferably at least 81%, at least 82, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to mammalian, in particular human FAHD1, preferably as described in PDB database code 1SAW and Manjasetty et al., Biol Chem, 2004, 385(10).

The present invention further provides a method for the prevention or treatment of aberrations of the energy metabolism of the nervous system, pancreas, kidney, liver, muscles or adipose tissue in a patient in need thereof, wherein FAHD1 or a homologue thereof is administered or its activity is otherwise increased so as to prevent or treat the aberration of the energy metabolism.

The present invention also provides a method for the prevention or treatment of aberrations of the energy metabolism of the nervous system, pancreas, kidney, liver, muscles or adipose tissue in a patient in need thereof, wherein FAHD1 or a homologue thereof is modified, in particular genetically modified, so as to prevent or treat the aberration of the energy metabolism.

For achieving the desired effects of FAHD1 on the energy metabolism in prevention or treatment, the FAHD1 protein or the FAHD1 gene should be provided in form of a pharmaceutical composition. A person skilled in the art will consider different administration routes. FAHD1 or a homologue thereof, which homologue comprises an amino acid sequence with at least 80% identity to FAHD1, could be applied as protein provided in a suitable pharmaceutical formulation preferably suitable for non-parental application, e.g. i.m., i.v., or s.c. injections. Additional means for improving cellular uptake and directing the protein towards the desired tissue could be applied. Regarding the intracellular distribution, a mitochondrial targeting sequence present in the FAHD1 protein sequence may enable the localization in mitochondria. A pharmaceutical composition according to the invention also comprises acceptable additives, such as a carrier, adjuvant, preservative or stabilizer.

In one aspect the invention provides a pharmaceutical composition comprising FAHD1 or homologue and a pharmaceutical acceptable additive.

The present invention also provides a pharmaceutical composition for use in a method for the prevention or treatment of aberrations of the energy metabolism of the nervous system, pancreas, kidney, liver, muscles or adipose tissue comprising a pharmaceutically effective amount of FAHD1 or a homologue thereof, optionally together with a pharmaceutically acceptable additive, such as a carrier, adjuvant, preservative or stabilizer.

In another aspect, the invention is related to a pharmaceutical composition comprising an oligonucleotide derived from a gene encoding for FAHD1 or a homologue thereof, and a pharmaceutical acceptable additive.

Gene therapy might be considered to achieve the effects of the FAHD1 enzyme. In these cases, an oligonucleotide derived from a gene encoding for FAHD1 or a homologue thereof, which homologue comprises an amino acid sequence with at least 80% identity to FAHD1, is provided in a suitable pharmaceutical composition. In this context, the oligonucleotide comprises at least in parts a sequence encoding for a FAHD1 protein. The oligonucleotide can be provided as double stranded DNA/RNA or a single stranded oligonucleotide. On the other hand gene therapy might be used for gene silencing or knockdown, wherein the knockdown is preferably a transient knockdown. In the context of FAHD1, the reduction of gene expression might have beneficial effects for use in treatment or therapy, preferably of aberrations of the energy metabolism. Silencing or knockdown of the FAHD1 gene expression results in reduced ODx activity, especially in mitochondria. Potential therapeutic mechanisms are discussed below in context of the beneficial effects of inhibition of FAHD1 activity via an inhibitor. Similar, a reduced FAHD1 activity level can be obtained via silencing of protein expression with compositions comprising an oligonucleotide with a FAHD1 antisense gene. A person skilled in the art can consider different variants how a gene silencing can be achieved; exemplary it may be achieved by using short interfering RNA (siRNA) which becomes effective via RNA interference. Thus, an oligonucleotide derived from a gene encoding for a FAHD1 protein or a homologue thereof, which homologue comprises an amino acid sequence with at least 80% identity to FAHD1, may also comprise a respective antisense oligonucleotide. It should be noted that in context of the gene therapy according to the invention, the term oligonucleotide may also comprise non-natural nucleotide analogues to improve pharmacodynamic or pharmacokinetic properties e.g. for enhancing stability of the oligonucleotide. To provide a pharmaceutical composition, the person skilled in the art would consider known means and methods for enabling gene therapy, for example consider protein transduction domains or viral vectors such as lentiviruses as means for providing a suitable pharmaceutical composition for gene therapy. Such means and methods as well as classical pharmaceutical carriers, adjuvants, preservatives or stabilizers are covered by the term “pharmaceutical acceptable additive”, which is comprised in a pharmaceutical composition according to the invention.

The present invention further relates to a method of decarboxylating an organic compound, preferably in vitro, comprising the steps of:

a) providing FAHD1 or a homologue thereof, which comprises an amino acid sequence with at least 80% identity to FAHD1, and a starting organic compound,

b) contacting FAHD1 or the homologue thereof and the starting organic compound with each other under conditions suitable to facilitate decarboxylation of the starting organic compound and

c) obtaining a decarboxylated organic compound.

In a preferred embodiment of the present invention, the starting organic compound used in step a) is oxaloacetate, which is decarboxylated in step b) to pyruvate. In particular, the decarboxylation in step b) occurs with a Vₘₐₓ of 0.1 to 0.3 μmol min⁻¹ mg⁻¹, in particular 0.2 μmol min⁻¹ mg⁻¹, and a Kₘ value of 20 to 40 μM, preferably 30 μM, in particular 32 μM.

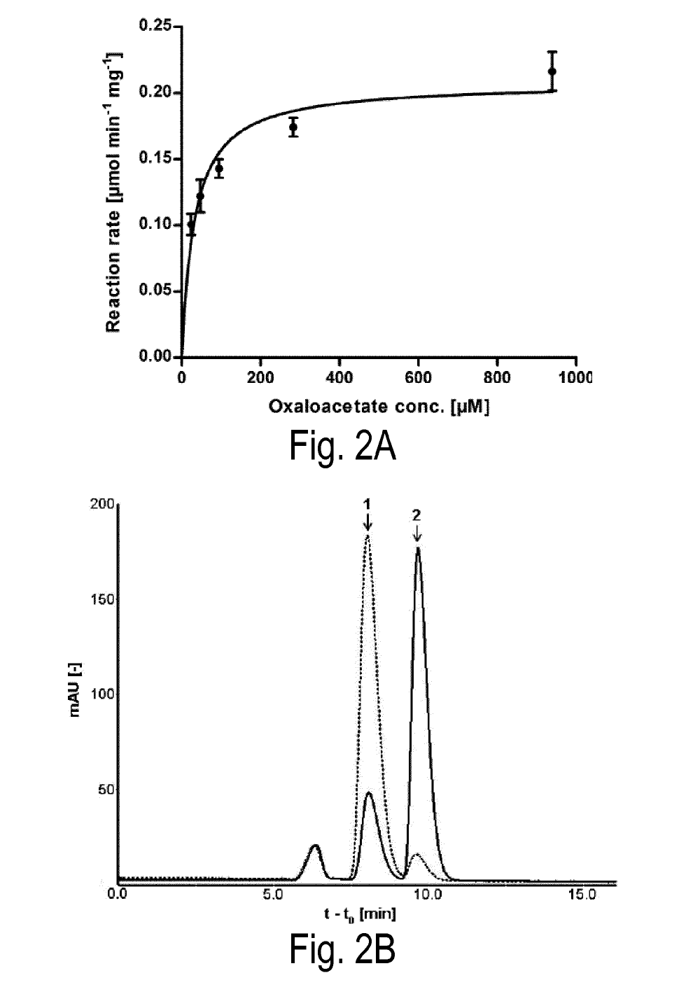

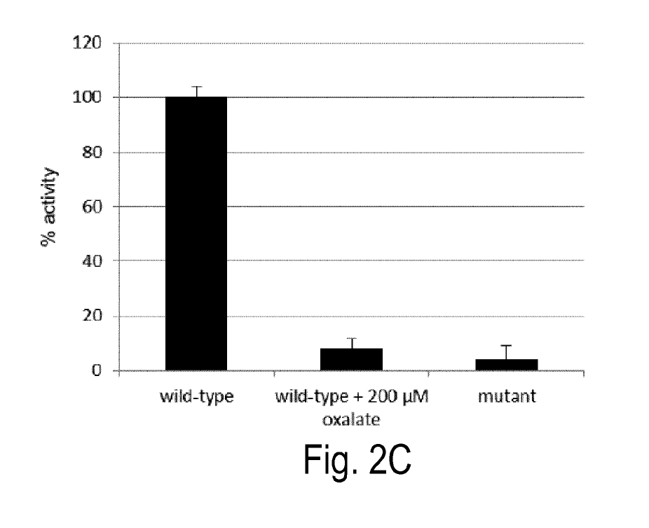

In a preferred embodiment of the method according to the present invention, the decarboxylase activity of FAHD1 or the homologue thereof can be modulated by a third compound. The third compound can have no effect on the decarboxylase activity of FAHD1 or act activating or inhibiting. In consequence, the method according to the invention may be adapted to identify modulators of the FAHD1 activity such as activators or inhibitors. A compound is considered as an inhibitor if a reduction in the activity of FAHD1 in comparison to the activity in absence of the investigated compound is observed. Exemplarily, the approach is described for the inhibitor oxalate as identified in Example 3. The results shown in FIG. 2 C provide oxalate as FAHD1 inhibitor.

In particular, the invention is related to a method of identifying a compound that inhibits the catalytic activity of FAHD1 or a homologue thereof comprising the steps of:

i) providing a candidate compound,

ii) contacting the FAHD1 or homologue thereof with the test compound under conditions allowing for the catalytic activity of FAHD1 or homologue thereof,

iii) determining whether the candidate compound inhibits the catalytic activity of FAHD1 or homologue thereof in comparison to the catalytic activity of FAHD1 or homologue thereof in absence of the candidate compound under same conditions.

A “candidate compound” can be a single compound or a mixture of different molecules, preferably the candidate compound is a defined organic compound, which should be investigated for its potential to inhibit FAHD1 activity. The method according to the invention can thus be used for screening of FAHD1 inhibitors, for example screening of a large number of candidate compounds. The person skilled in the art will consider providing the candidate compound in a predefined concentration. Preferably different concentrations of the candidate compound are provided for the method of identification of inhibitors, which also allows characterizing the effect of the candidate compound quantitatively. Step ii) is achieved for example in that an assay buffer used for determining the catalytic activity is supplemented by the candidate compound.

The catalytic activity of FAHD1 can be determined by different approaches such as the oxaloacetate decarboxylase assay as disclosed in example 3. As shown in this example, time dependent reduction of the substrate's concentration can be monitored by e.g. UV absorption of oxaloacetate at 255 nm. As also disclosed, oxaloacetate decarboxylase assay should be corrected for autodecarboxylation of the substrate as measured in absence of FAHD1. Alternatively, acylpyruvase activity could be monitored for assessing the catalytic activity of FAHD1 and evaluating a candidate compound. It is expected that a compound inhibiting acylpyruvase activity of FAHD1 is also inhibiting oxaloacetate decarboxylase activity as the teaching of the present invention suggests that the same catalytic center is responsible for both reactions. Inhibition of FAHD1 by the candidate compound will be reflected in elevated substrate concentration in comparison to the substrate concentration in absence of the candidate compound under same conditions. Alternatively, inhibition is characterized in reduced product concentration in comparison to the product concentration in absence of the candidate compound under same conditions. For the latter case, an indirect assay may be applicable wherein the FAHD1 product pyruvate is further transformed to lactate by NADH dependent lactate dehydrogenase (LDH). Readout is based on the concentration of NADH at 335 nm. The time-dependent NADH signal is directly proportional to FAHD1 inhibition. The higher the FAHD1 activity the more pyruvate is converted and the lower is the NADH signal. Inhibition leads to less pyruvate and in consequence less NADH is transformed, resulting in higher absorbance signals. Following conditions are recommended: 200 μM NADH, 100 μM oxaloacetate, 0.1 U LDH, 12 μg FAHD1 in 1 ml.

In an embodiment of the method for identifying a compound that inhibits the catalytic activity of FAHD1 or a homologue thereof, the catalytic activity is determined by a difference in concentration of
a) a catalytic substrate of FAHD1, wherein inhibition of the catalytic activity is indicated by a higher substrate concentration in comparison to the substrate concentration in absence of the candidate compound under same conditions or
b) a catalytic product of FAHD1, wherein inhibition of the catalytic activity is indicated by a lower product concentration in comparison to the product concentration in absence of the candidate compound under same conditions.

In a preferred embodiment the catalytic substrate for which a difference in concentration is determined may be oxaloacetate. In another preferred embodiment the catalytic product for which a difference in concentration is determined may be pyruvate.

In one aspect the invention provides a kit for identification of a FAHD1 inhibitor comprising FAHD1 or a homologue thereof, a substrate of FAHD1, and an instruction to determine catalytic activity of FAHD1.

Such a kit allows for testing potential inhibitors according to an assay as described above and enables the identification of inhibitors of FAHD1. Preferably the kit initially provides the FAHD1 or a homologue thereof separate from the substrate. The substrate is a compound that is transformable by FAHD1 or a homologue thereof and allows detection of FAHD1 catalytic activity. The instructions disclose a protocol optimized for catalytic activity of FAHD1. The instructions may preferably also to include guidelines for control experiments and guidelines to classify a candidate compound as FAHD1 inhibitor.

The inventors show as an example that sodium oxalate, the salt of the dicarboxylic oxalic acid, is an inhibitor of FAHD1. Inhibitors of FAHD1, which are to be understood as compounds inhibiting the FAHD1 catalytic activity, i.e. the oxaloacetate decarboxylase activity, are compounds with desirable properties. They are suitable tools for investigation of FAHD1 enzyme activity and its biological role. More important, given the function of FAHD1 as oxaloacetate decarboxylase, inhibiting the enzymatic activity of FAHD1 provides means and methods to increase the concentration of oxaloacetate in mammals, in particular in mitochondria of mammals.

Thus, the characterization of FAHD1 as an oxaloacetate decarboxylase enables to specifically use an inhibitor, in particular an in vivo inhibitor, of FAHD1, and thus to modulate, in particular increase or decrease, preferably increase, the level of oxaloacetate and/or the activity of the counteracting enzyme PC in eukaryotic, in particular mammalian, cells. In particular, the present invention identifies and enables the use of the inhibition of the oxaloacetate decarboxylase activity of FAHD1 in mammals, in particular humans.

In particular, the present teaching relates to the use of inhibitors the oxaloacetate decarboxylase activity of FAHD1 opening up various treatment options for energy metabolism related diseases involving the FAHD1/PC metabolic module, in particular kidney, liver, pancreatic and neurological diseases, diabetes, metabolic syndrome or obesity.

As described above, FAHD1 counteracts the enzyme PC, which is involved in regulation of carbohydrate and lipid metabolism. Based on this link a therapeutic potential for inhibitors of FAHD1 is expected.

For example in diabetes, continued high glucose plasma levels are associated with a decrease of PC expression in pancreas. Reduced PC level are believed to play an important role in decline of pancreatic β-cells and decompensated diabetes mellitus type II, a very important aberration of the energy metabolism with severe clinical effect. Based on the finding of the present invention, it is believed that reduced activity of the PC can be balanced by inhibiting the FAHD1. Compounds inhibiting FAHD1 identified by a method according to the invention are believed to be potential candidates for use in treatment or prevention of diabetes mellitus type II.

Moreover, it was shown that FAHD1 knock-out mice showed slightly lower fat mass and fat gain under high fat diet was reduced in comparison to wild-type mice. Similar effects could be expected for reduced FAHD1 activity by application of FAHD1 inhibitors. Thus, inhibition of FAHD1 could be promising for use in treatment or prevention of for example obesity or metabolic syndrome.

Thus, inhibitors of FAHD1 could be suitable for use in treatment or therapy of disease such as aberration in the energy metabolism of the nervous system, pancreas, kidney, liver, muscles or adipose tissue. Especially such aberrations are linked to metabolic pathways like the tricarboxylic acid cycle, gluconeogenesis, de novo fatty acid synthesis, glyceroneogenesis, insulin secretion and de novo synthesis of glutamate. Aberration of the energy metabolism may be various diseases or conditions such as type 2 diabetes mellitus, obesity, hypercholesterolemia, epilepsy, attention deficit hyperactivity disorder (ADHD), Parkinson's disease, Alzheimer's disease, focal cerebral ischemia (stroke), lactic acidosis, psychomotor deficiencies or death in infancy.

The present invention further discloses a method for the prevention or treatment of aberrations of the energy metabolism of the nervous system, pancreas, kidney, liver, muscles or adipose tissue is provided, wherein the activity of FAHD1 or a homologue thereof is modified, in particular inhibited, in a subject in need thereof by administering a modifying, in particular inhibiting, amount of an effector, in particular inhibitor, to the subject in need thereof.

In another aspect, the present invention provides a method of treatment or prevention of a disease involving an aberration of the energy metabolism. The method comprises the step of administering to a patient in need thereof
i) FAHD1 or a homologue thereof, which comprises an amino acid sequence with at least 80% identity to FAHD1, or
ii) an oligonucleotide derived from a gene encoding for FAHD1 or a homologue thereof.

Further preferred embodiments are the subject-matter of the dependent claims.

The invention will now be described in more detail by the non-limiting examples.

The figures show:

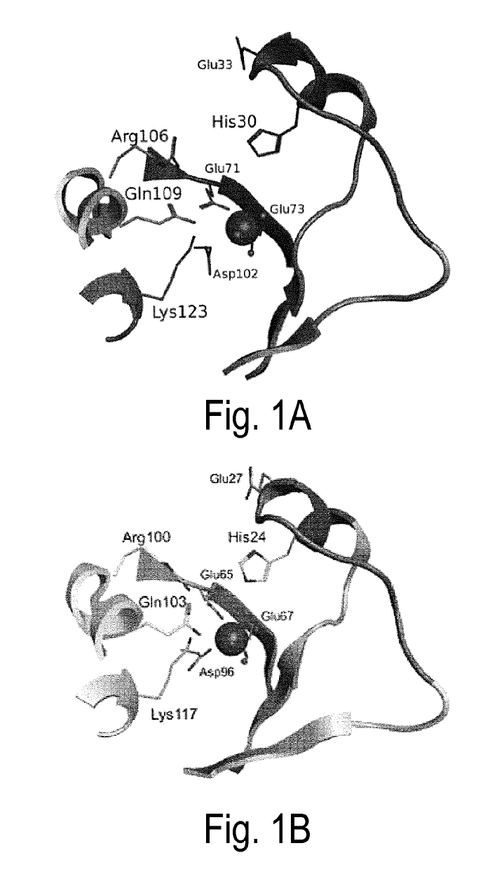

FIG. 1 shows the active site geometry of human FAHD1 (A) and FAHD-1 of C. elegans (B). By computational loop modelling, a closed structure of FAHD1 (A) was established where His30 and Glu33 complete the active site. The lid region carrying the catalytic histidine and an activating glutamate residue is closed upon binding of the inhibitor oxalate (shown as sticks). The model of the closed lid region in FAHD1 completes the active site of FAHD1 around the magnesium ion (large sphere). Conserved residues around the active site are shown as sticks and labelled. Secondary structure elements are represented as cartoons. Two water molecules (small spheres) are co-crystallized in FAHD1 at the binding site of the oxalate, suggesting a similar binding mechanism. (B) The nematode protein, referred to as FAHD-1, contains 48% identical amino acids relative to human FAHD1, shows high amino acid sequence similarity in the presumed catalytic centre, and computational modelling revealed a structure of the catalytic centre highly similar to human FAHD1.

FIG. 2 shows the characterization of in vitro ODx activity of wild-type and mutant FAHD1. (A) Effect of substrate concentration on ODx reaction rate in presence of purified FAHD1, determined by photometric analysis at RT. Nonlinear regression analysis of Michaelis-Menten kinetics was performed with Prism5 software (GraphPad Software). Data are represented as mean±SD (n=3). (B) HPLC analysis of oxaloacetate breakdown in presence (solid line) and absence (dashed line) of purified FAHD1. Retention times for oxaloacetate (1) and pyruvate (2) standards are indicated by arrows. (C) ODx activity of wild-type FAHD1 in presence/absence of 200 μM oxalate, and of mutant FAHD1. Data are represented as mean±SD (n=3).

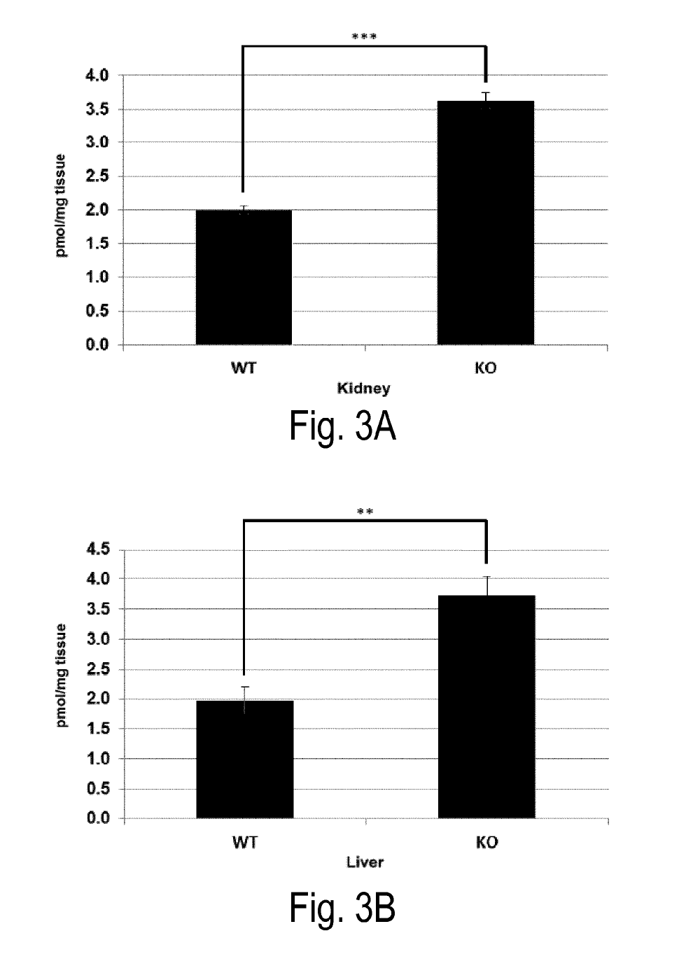

FIG. 3 shows the comparison of oxaloacetate levels in organs of wild-type (WT) and FAHD1 knockout mice (KO). (A+B) Oxaloacetate levels determined in kidney (A) and liver (B) extracts of 3-month-old female wild-type and FAHD1 knockout mice. Data are represented as mean±SD (n=3).

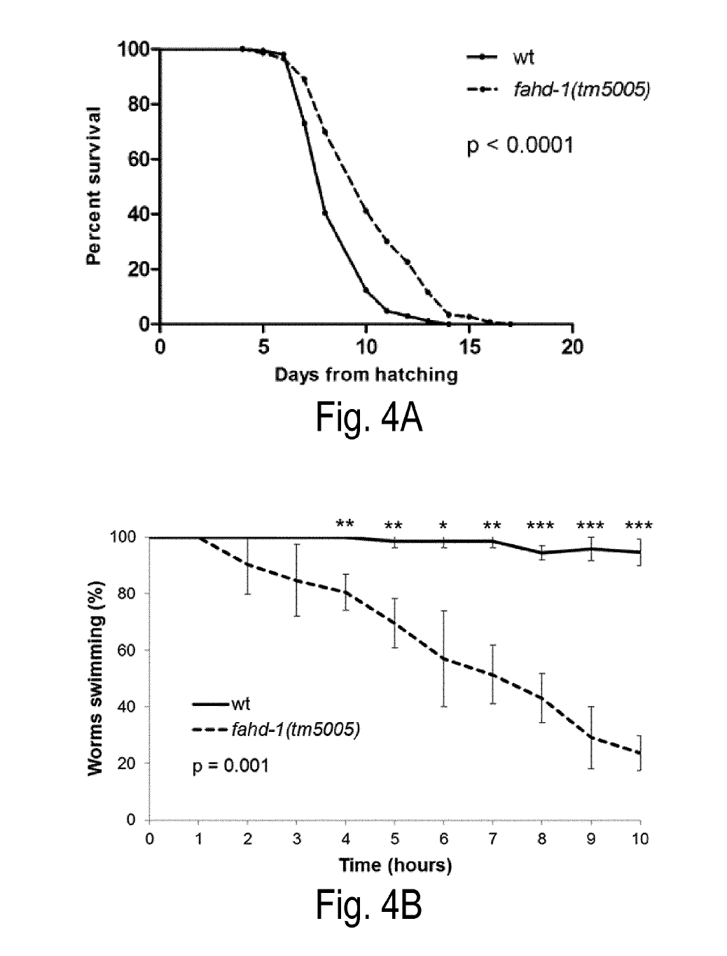

FIG. 4 shows the phenotypic characterization of fahd-1 (tm 5005) C. elegans mutants. (A) Survival curves at 25° C., showing percentage of animals remaining alive over time. (B) Worms were subjected to a swimming assay in M9 buffer at 20° C. The percentage of animals still swimming is plotted against time (n=24).

FIG. 5 shows the putative role of FAHD1 in central metabolism as an antagonist of pyruvate carboxylase (PC).

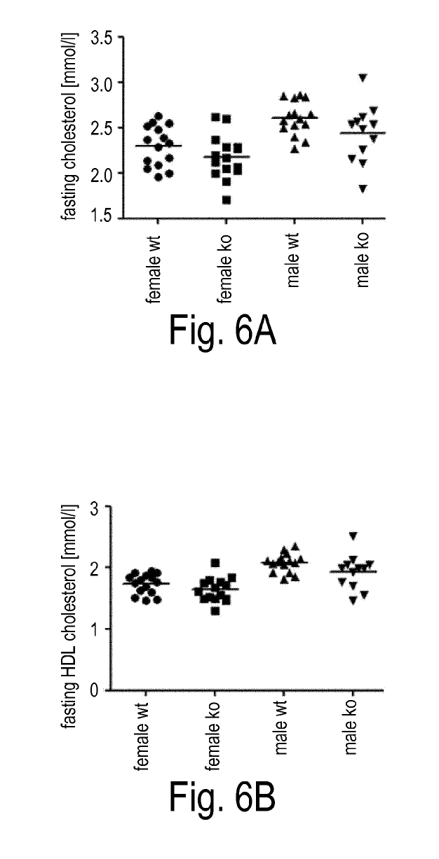

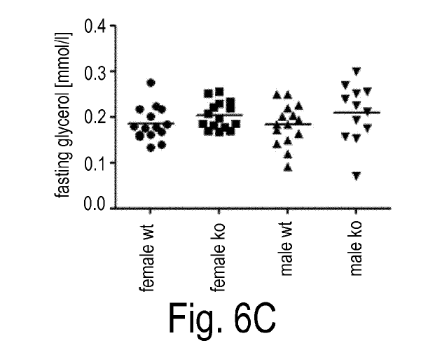

FIG. 6 shows the effect of FAHD1 depletion on the plasma levels of cholesterol (A), HDL-cholesterol (B), and gycerol (C) in female and male FAHD1 knockout mice (ko) and wild-type (wt) mice after food withdrawal overnight (fasting).

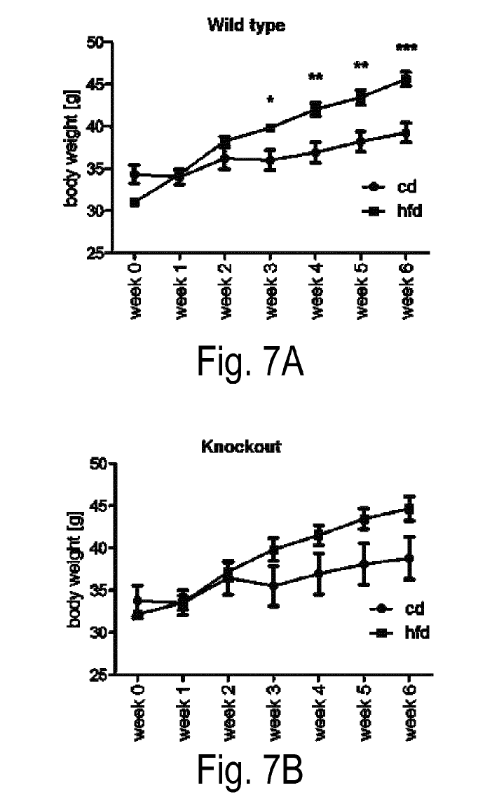

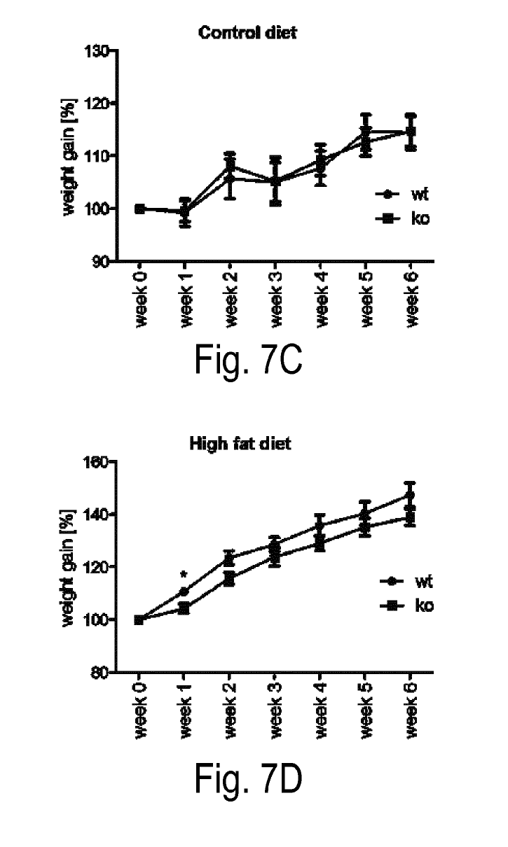

FIG. 7 shows body weight development of wild-type mice (A) and FAHD1 knockout mice (B) under control diet (cd, circles) and high fat diet (hfd, squares). Relative weight gain under control diet (C) and high fat diet (D) is shown for wild-type (circles) and FAHD1 knockout mice (squares).

### EXAMPLES

### Example 1: Modelling of FAHD1 Active Site

Method:

The X-ray structure of human FAHD1 (PDB database code 1SAW) (Manjasetty et al., Biol Chem, 2004, 385(10)) lacks 11 highly flexible residues next to the active site (Asp29 to Leu39). This region is constructed by using the ‘Loop modeller’ tool of MOE. To allow the loop region to extend into the active site—as expected for a closed lid conformation—four water positions were deleted in the active site: HOH314, HOH316, HOH356, HOH359. The missing loop was defined between Val21 and Val43 to identify potential loop candidates from the PDB. A tolerant maximal walk step of 5 amino acids allowed varying potential anchoring points between residue 16 and residue 48. All other parameters were kept as default. 94 structured loop regions were identified as potential templates with these parameters. The best scored candidate from the putative FAH protein from Yersinia pestis (PDB code 3S52) was used as template structure for loop construction. The parameters for refined loop modelling included an adaption of the environment by side chain repacking (default parameters).

Results:

By computational loop modelling, a closed structure of FAHD1 is established where His30 and Glu33 complete the active site. The lid region carrying the catalytic histidine and an activating glutamate residue is closed upon binding of the inhibitor oxalate (shown as sticks). The model of the closed lid region in FAHD1 completes the active site of FAHD1 around the magnesium ion (large sphere). Conserved residues around the active site are shown as sticks and labelled. Secondary structure elements are represented as cartoons. Two water molecules (small spheres) are co-crystallized in FAHD1 at the binding site of the oxalate, suggesting a similar binding mechanism (see FIG. 1A).

### Example 2: Bacterial Recombinant Expression and Purification of FAHD1 and FAHD1mut

N-terminally His- and S-tagged versions of human wild-type FAHD1 and a double mutant (Asp102Ala, Arg106Ala) were recombinantly expressed in E. coli and purified as reported previously (Pircher et al., J Biol Chem, 2011, 286(42)).

### Example 3: Oxaloacetate Decarboxylase Assay

Method:

For measurement of oxaloacetate decarboxylase rates, 1 ml samples of 25 μM to 1 mM oxaloacetate in assay buffer (50 mM Tris-HCl, 100 mM KCl, 1 mM MgCl2, pH 7.4) containing purified FAHD1, FAHD1mut (3-60 μg, depending on substrate concentration) or no enzyme were prepared. Samples were incubated at room temperature and analysed in regular time intervals by measuring absorbance (infinite M200, Tecan) at 255 nm (ε=1070 M⁻¹ cm⁻¹) in disposable UV-cuvettes (Brand). Reaction mixtures containing no substrate were used as blank. For the oxalate inhibitor assay, the sample buffer was supplemented with 200 μM sodium oxalate. All rates were corrected for auto-decarboxylation under assay conditions.

Results:

The recombinantly expressed wild-type and mutant FAHD1 proteins (example 2) were tested in the above photometric assay suitable for monitoring the breakdown of oxaloacetate. The purified wild-type enzyme was able to degrade oxaloacetate with a Vmax of 0.21 μmol min⁻¹ mg⁻¹ and a Km value of 32 μM (FIG. 2A). To further characterize the ODx activity inherent to FAHD1, oxalate was used, an inhibitor of oxaloacetate decarboxylase Cg1485. Indeed, oxalate potently inhibited oxaloacetate decarboxylation by FAHD1, with an IC50 value of about 20 μM (FIG. 2C).

### Example 4: Analysis of Oxaloacetate Decarboxylase Reaction by HPLC

Method:

1 ml assay buffer containing oxaloacetate (1 mM) and purified recombinant FAHD1 protein (120 μg) was incubated at room temperature (RT) for 30 min. A control lacking FAHD1 was incubated analogously. The conversion mixture and control of the FAHD1 reaction were analysed by high performance liquid chromatography (HPLC) using an ÄKTA purifier system (GE Healthcare) equipped with a Bio-Rad Aminex HPX-87H column (300×7.8 mm). Detection was at 210 nm. 84 μl of sample were injected and eluted with 5 mM H2SO4 as the eluent at a flow rate of 0.5 ml min⁻¹ at RT. Identification of peaks was based on the characteristic retention times of high purity standards (>99%) of oxaloacetate and pyruvate.

Results:

The above HPLC analysis, after incubation of the substrate in presence or absence of the purified enzyme, confirmed the conversion of oxaloacetate to pyruvate in presence of FAHD1 (FIG. 2B). Incubation without the enzyme only yielded a minor amount of pyruvate due to auto-decarboxylation, whereas the catalytically dead mutant enzyme displayed only residual ODx activity (FIG. 2C).

### Example 5: Generation of a FAHD1^{-/-} knockout mouse

Method:

F2 generation C57BL/6 mice heterozygous for a LoxP-flanked FAHD1 gene were established by inGenious Targeting Labs. FAHD1^{-/-} mice were generated by crossing FAHD1^{flox/+} with Cre^{0/+} transgenic mice, followed by outcrossing of Cre alleles and crossing of FAHD1^{-/+} mice. The knockout was verified by PCR and immunoblot. Care of experimental animals was in accordance with guidelines for mouse work at Universität Innsbruck.

Results:

A cohort of 15 male wild-type mice and 13 male knockout mice as well as 15 female wild-type and 15 female knockout mice was generated. To determine body composition, body mass and fat mass were investigated using time domain nuclear magnetic resonance (TD-NMR). The comparison of body mass and fat mass between wild-type and knockout mice displayed a slightly reduced body weight as well as decreased fat mass to some extent in male knockout mice compared to their wild-type controls. This trend is not that pronounced in female mice. Based on these findings, body composition of wild-type and knockout mice did not differ significantly. But a slight significant genotype dependent difference was observed, when taking female and male mice together displaying lower body mass of knockout mice (p=0.041). Although fat mass of wild-type and knockout mice does not differ significantly, still there is a trend towards lower fat mass in knockout mice. In addition to body composition, body surface temperature was measured to examine any discrepancies in overall body temperature. A trend to increased body temperature of male knockout mice compared to wild-type was found (less so for female mice) (p=0.091).

It was of interest to examine whether knockout mice display a different pattern in parameters involved in energy metabolism. For this purpose blood plasma parameters were controlled in mice that were fasted overnight. The screen was focusing on cholesterol, HDL-cholesterol and glycerol levels, which play an important role in lipid metabolism and as structural membrane components. Furthermore these parameters can be used as indicator for the diagnosis of the metabolic syndrome. Serum cholesterol as well as HDL-cholesterol levels of female and male knockout mice (separately) showed a trend towards decreased cholesterol and HDL-cholesterol levels (FIGS. 6A and 6B). In contrast, glycerol levels of knockout mice were slightly increased in both sexes (FIG. 6C). When comparing wild-type and knockout mice of both sexes together HDL-cholesterol and cholesterol levels were significantly decreased in knockout animals compared to control mice (p=0.018 cholesterol; p=0.016 HDL-cholesterol).

A cohort of 10 male wild-type and knockout mice each at the age of 7 months was divided into 4 groups. One group of wild-type mice obtained food containing high fat and the other was just fed with food usually used for animal keeping. The same procedure was applied for knockout mice. This special feeding was conducted over a time span of 6 weeks, including weekly measurement of body weight. As expected wild-type mice on a high fat diet showed a continuously increasing body weight differential over a time span of 3 weeks when compared to mice fed with control food. Elevated body weight was also observed in knockout mice even though the difference to control-fed mice was not significant (FIG. 7A). When concentrating on the weekly weight gain of mice fed with control diet, no genotype dependent discrepancy could be observed. Wild-type mice significantly increased their body weight 1 week upon feeding a high fat diet compared to Fand1 knockout animals, keeping this trend until the end of the experiment after 6 weeks (FIG. 7B).

### Example 6: FAHD1 Western Blot

Method:

Frozen mouse organs were homogenized in PBS supplemented with protease inhibitors (1 Complete Mini EDTA-free tablet per 10 ml, Roche). Lysate supernatants (30 μg total protein) were separated by SDS-5 PAGE (12.5% acrylamide) and blotted onto a PVDF membrane. Rabbit monoclonal anti-mouse FAHD1 antibody (purpose-made, 14 μg/ml) and anti-rabbit HRP-conjugated secondary antibody (Dako P0399, 1:2,500) were applied by standard Western blot protocol. α-tubulin antibody (Sigma T5168, 1:10,000) was used for loading control with an anti-mouse HRP-conjugated secondary antibody (Dako P0447, 1:20,000). Detection was achieved by ECL Prime (GE Healthcare).

Results:

In mice, FAHD1 is predominantly expressed in kidney and liver.

### Example 7: Analysis of Oxaloacetate Levels in Mouse Tissue

Method:

3-month-old female C57BL/6 mice (wild-type and FAHD1^{-/-} littermates) were sacrificed by cervical dislocation and the desired organs (kidney and liver) were immediately excised and shock-frozen in liquid nitrogen. Frozen organs were homogenized in ice-cold 5% perchloric acid (1 ml per 100 mg tissue) and assayed according to the method described by Parvin et al. (Anal Biochem, 104, 1980). Briefly, after hydrolysis of endogenous acetyl-CoA and subsequent neutralization, a 25 μl aliquot was included in a 200 μl reaction containing 0.6 units citrate synthase (Sigma) and 3 pmol [³H]acetyl-CoA (39 nCi, Moravek Biochemicals) to transform endogenous oxaloacetate into [³H]citrate. After adsorption of unreacted [³H]acetyl-CoA to activated charcoal (Sigma), samples were measured in a liquid scintillation counter (LS 6500, Beckman).

Results:

Metabolites were extracted from both the kidney and the liver of FAHD1^{-/-} Mice and wild-type littermates as described above. The concentration of oxaloacetate in these extracts was determined with the above enzymatic assay utilizing the reaction with ³H-labelled acetyl-CoA to form citrate. The concentration of oxaloacetate was significantly increased in both the kidney (FIG. 3A) and the liver (FIG. 3B) of FAHD1^{-/-} mice, indicating that oxaloacetate is indeed a relevant in vivo substrate for FAHD1 in mice.

### Example 8: Modelling of Nematode FAHD-1 Structure

Method:

A homology model was generated to compare the structure of FAHD-1 from C. elegans based on the sequence of ZK688.3 (NP 498715.1). As expected, sequence search revealed the FAH domain containing structures as suitable templates. Sequence identity is 46.3% for FAHD1 (PDB code 1SAW), with a similarity of 64.5%. Although this structure is the closest in sequence, the FAH protein from Yesinia pestis C092 (PDB code 3S52) was selected as template, having a better resolution and a completely resolved structure for chain A (closed and structured lid). The template and the target sequence share 39.7% sequence identity. The model was generated with MOE homology modelling tool (Chemical Computing Group, MOE release 2013.08) with chain A and D of 3S52 as templates for a dimer model using the force field option Amber12EHT. The model had unexpected cis amid configurations for Arg8 in chain A and B as well as Lys13 in chain A, which are solvent exposed or in the dimer interface respectively. They are associated with outliers in the Ramachandran plot for the neighboring Asn9 in chain B and Lys13. Additionally the distal residue Asn147 in chain B and Pro135 in both subunits have suspicious backbone configurations. However, the binding site shows no parameters indicating quality issues in the model structure. In the active site, side chains orientations of Arg100 and Glu65 were manually adapted. The initial model was complemented by water positions and co-crystallized ions from the structure of human FAHD1 (PDB code 1SAW) and not the template as the latter does not include the magnesium ion in the active site. Eight individual water molecules forming too close contacts with the model were removed. To allow water and active site adaptions to the magnesium ion, the assembly was energy minimized in several steps with decreasing positional restraints on the atoms.

Results:

The nematode protein, referred to as FAHD-1, contains 48% identical amino acids relative to human FAHD1, shows high amino acid sequence similarity in the presumed catalytic centre, and computational modelling revealed a structure of the catalytic centre highly similar to human FAHD1 (FIG. 1B).

### Example 9: FAHD-1 Mutant Nematodes

The fahd-1(tm5005) mutant C. elegans strain was obtained from the Japanese ‘National Bioresource Project’ for the experimental animal ‘Nematode C. elegans’. This mutant carries a 236 bp deletion in the fahd-1 gene that removes exon 2 and leads to a frameshift in exon 3, and was confirmed by genomic PCR and Western blot, using a peptide-specific rabbit polyclonal antibody raised against FAHD-1. The strain was backcrossed six times to N2 Bristol wild-type C. elegans to eliminate any possible second-site mutations.

### Example 10: C elegans Lifespan Analysis

Method:

Lifespan assays were conducted according to established methods (Artal-Sanz & Tavernarakis, 2009, Nature 461) at 25° C. Animal populations were synchronized by allowing adult hermaphrodites to lay eggs for a limited time interval (2 hours) on NGM plates seeded with E. coli OP50. These synchronized embryos developed into adulthood under controlled conditions and were then spread on fresh plates (20 worms per plate), totaling 150-200 individuals per experiment. The day of egg harvest was defined as t=0. Animals were moved to fresh plates every 1-2 days and examined daily for touch-provoked movement and pharyngeal pumping. Worms dying due to internally hatched eggs, an extruded gonad, or desiccation due to leaving the agar were censored and incorporated as such into the data set. Each survival assay was repeated at least three times. Survival curves were generated according to the product-limit method of Kaplan and Meier. Differences between survivals and p values were evaluated via the log-rank (Mantel-Cox) test. The Prism software package (GraphPad Software) was used to carry out statistical analysis and to determine lifespan values.

Results:

Deletion of FAHD-1 resulted in a significant extension of lifespan (FIG. 4A). The effect was most pronounced in worms grown at 25° C. In addition, fahd-1(tm5005) mutant animals displayed severe locomotion defects and a strongly reduced capacity for physical activity, as revealed by an endurance swimming test in liquid (FIG. 4B). Together, these results establish FAHD-1 as a novel important determinant of mitochondrial metabolism, the deletion of which impairs mitochondrial function and physical fitness in nematodes.

### Example 11: Assessment of Mitochondrial Membrane Potential in C. elegans

Method:

L4 larvae of wild-type and fahd-1(tm5005) C. elegans were placed on NGM plates seeded with E. coli OP50 and containing 100 nM tetramethylrhodamine ethyl ester (TMRE, Sigma). After overnight incubation at 20° C. the worms were imaged within 3 minutes after anaesthetizing with 10 mM levamisol hydrochloride VETRANAL (Fluka), using a Nikon Eclipse TE300 microscope.

Results:

A significant reduction of mitochondrial membrane potential in fahd-1(tm5005) mutants was observed relative to wild-type worms, indicating that FAHD-1 is required for proper function of mitochondria in nematodes.

1. A method for the prevention or treatment of a disease comprising administering to a patient in need thereof FAHD1 or a homologue thereof, which comprises an amino acid sequence with at least 80% identity to FAHD1.

2. A method for the prevention or treatment of aberrations of the energy metabolism of the nervous system, pancreas, kidney, liver, muscles or adipose tissue comprising administering to a patient in need thereof FAHD1 or a homologue thereof, which comprises an amino acid sequence with at least 80% identity to FAHD1.

3. The method according to claim 2, wherein the aberration of the energy metabolism is type 2 diabetes mellitus, obesity, hypercholesterolemia, metabolic syndrome, epilepsy, attention deficit hyperactivity disorder (ADHD), Parkinson's disease, Alzheimer's disease, focal cerebral ischemia (stroke), lactic acidosis, psychomotor deficiencies, mental disorder or death in infancy.

4. The method according to claim 2, wherein the aberration of the energy metabolism is the reduction of the oxaloacetate concentration.

5. The method according to claim 3, wherein the aberration of the energy metabolism is metabolic syndrome or type 2 diabetes mellitus.

6. A pharmaceutical composition comprising FAHD1 or a homologue thereof, which comprises an amino acid sequence with at least 80% identity to FAHD1, and a pharmaceutical acceptable additive.

7. A pharmaceutical composition comprising an oligonucleotide derived from a gene encoding for FAHD1 a homologue thereof, which comprises an amino acid sequence with at least 80% identity to FAHD1, and a pharmaceutical acceptable additive.

8. A method of decarboxylating an organic compound, comprising the steps of:
a) providing FAHD1 or a homologue thereof, which comprises an amino acid sequence with at least 80% identity to FAHD1, and a starting organic compound,
b) contacting FAHD1 or the homologue thereof and the starting organic compound with each other under conditions suitable to facilitate decarboxylation of the starting organic compound and
c) obtaining a decarboxylated organic compound.

9. The method according to claim 8, wherein the starting organic compound used in step a) is oxaloacetate, which is decarboxylated in step b) to pyruvate.

10. A method of identifying a compound that inhibits the catalytic activity of FAHD1 or a homologue thereof, which comprises an amino acid sequence with at least 80% identity to FAHD1, comprising:
i) providing a candidate compound,
ii) contacting FAHD1 or the homologue thereof with the test compound under conditions allowing for the catalytic activity of FAHD1,
iii) determining whether the candidate compound inhibits the catalytic activity of FAHD1 or homologue thereof in comparison to the catalytic activity of FAHD1 or homologue thereof in absence of the candidate compound under same conditions.

11. The method according to claim 10, wherein the catalytic activity is determined by a difference in concentration of
a) a catalytic substrate of FAHD1, wherein inhibition of the catalytic activity is indicated by a higher substrate concentration in comparison to the substrate concentration in absence of the candidate compound under same conditions or
b) a catalytic product of FAHD1, wherein inhibition of the catalytic activity is indicated by a lower product concentration in comparison to the product concentration in absence of the candidate compound under same conditions,

12. The method according to claim 11, wherein the catalytical substrate is oxaloacetate, or the catalytical product is pyruvate.

13. A kit for identification of a FAHD1 inhibitor comprising FAHD1 or a homologue thereof, which comprises an amino acid sequence with at least 80% identity to FAHD1, a substrate of FAHD1, and an instruction to determine catalytic activity of FAHD1.

14. A method of treatment or prevention of a disease involving an aberration of the energy metabolism comprising administering FAHD1 or a homologue thereof, which comprises an amino acid sequence with at least 80% identity to FAHD1, or comprising administering an oligonucleotide derived from a gene encoding for FAHD1 or a homologue thereof, to a patient in need thereof.
